(19) United States
(12) Patent Application Publication
(10) Pub. No.: US 2004/0078081 A1
(43) Pub. Date: Apr. 22, 2004

(54) Bioresorbable components and methods for spinal arthroplasty

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/657,914

(22) Filed: Sep. 9, 2003

Related U.S. Application Data
(60) Provisional application No. 60/409,207, filed on Sep. 9, 2002.

(51) Int. Cl. A61F 2/44

(52) U.S. Cl. 623/17.16

(57) ABSTRACT

This invention broadens the method and apparatus to include the use of any biosorbable implant to temporarily immobilize the spine following any spinal arthroplasty. In addition to screws, implants may include rods, plates, and other elements, for the anterior or posterior portion of the spine.

### REFERENCE TO RELATED APPLICATION

This application claims priority from U.S. Provisional Application Serial No. 60/409,207, filed Sep. 9, 2002, the entire content of which is incorporated herein by reference.

### FIELD OF THE INVENTION

This invention relates generally to spinal surgery and, in particular, to bioresorbable components and methods to temporarily immobilize the spine following any spinal arthroplasty.

### BACKGROUND OF THE INVENTION

Prosthetic implants have historically been constructed from materials such as metal or ceramic, which remain in the body following a procedure. In cases where the implant is performing a temporary task, however, the use of such permanent fixtures may not be warranted. If tissues no longer need the structural support of an implant, permanent implants run the risk of being rejected by the body, migrating to undesirable locations and/or inflicting damage on surrounding tissue.

For such ‘temporary’ applications, bioresorbable implants are being developed which naturally resorb after they are no longer needed. As such, post-surgical complications associated with permanent implants may be dramatically reduced.

Bioresorbable implants are typically made from molecules similar to those found in the human body and resorb after the tissue is healed. Bioresorbable polymer implants, for example, typically use lactic acid in combination with lactic acid derivatives known as lactides. The resulting copolymers, generally referred to as polylactides, maintain their strength during the healing process, then gradually break down into lactic acid molecules through hydrolysis. Other bioresorbable materials are also available, as disclosed in U.S. Pat. No. 5,571,193, incorporated herein by reference.

Different resorption rates are beneficial for various surgical applications. For example, rapidly healing tissue (pediatric) may require an implant with a faster resorption rate than an implant designed for adult tissue that may experience slower or impaired healing. The resorption rates are controlled through material selection and fabrication

Bioresorbable implants may be shaped into various bodies, and thin-film versions are also available. My U.S. Pat. No. 6,344,058 describes the use of bio-resorbable screws to temporarily immobilize the spine after transplantation of a disc and vertebral endplates. U.S Pat. Nos. 6,019,792; 6,179,874 and 6,440,168 teach the use of bioresorbable components that fit between the endplates of artificial disc replacements (ADRs) to limits initial ADR motion. However, no comprehensive bioresorbable system has been developed to temporarily immobilize an entire level of the spine following any spinal arthroplasty.

### SUMMARY OF THE INVENTION

This invention broadens the method and apparatus to include the use of any biosorbable implant to temporarily immobilize the spine following any spinal arthroplasty. In addition to screws, implants may include rods, plates, and other elements, for the anterior or posterior portion of the spine.

### BRIEF DESCRIPTION OF THE DRAWING

FIG. 1 is a diagram of a preferred embodiment of the invention.

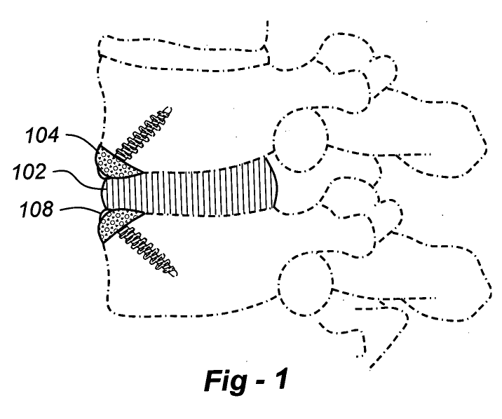

### DETAILED DESCRIPTION OF THE INVENTION

A preferred embodiment of the invention is depicted in the Figure, which shows an allograft disc 102 between upper and lower vertebra. While contact is made to patient endplates posteriorally, allograft endplates 104 are used anteriorally, these being held in place with screws 108.

The temporary instrumentation holds the vertebrae and the prosthetic or natural arthroplasty device until the soft tissues surrounding the spine heal. The instrumentation would also facilitate the vertebrae to heal to the arthroplasty device. For example, immobilization may help bone grow into the arthroplasty device.

These bio-resorbable devices would not require removal, as they would dissolve after healing has occurred. Spinal motion across the arthroplasty device would occur after the biosorbable instrumentation dissolves. The resorbable component may also decrease the amount of scar tissue that forms over the spine.

Limited ADR motion may also be allowed by the bioresorbable component. For example, the amount of ADR motion could be controlled by the flexibility of the bioresorbable component. The flexibility of the component could be controlled by the modulus of elasticity of the material and the thickness of the component. The duration of the limited mobility of the ADR could also be controlled by the rate of resorption of the material.

The invention is not limited in terms of bioresorbable materials, and may use lactides or any other existing materials disclosed in the patents referenced herein as well as any appropriate yet-to-be developed polymeric or non-polymeric materials.

I claim:

1. In spinal arthroplasty, the improvement comprising:
one or more bio-resorbable components to hold a vertebrae and a prosthetic or natural arthroplasty device in place until soft tissues surrounding the spine heal.

2. The improvement of claim 1, wherein the components include a rod, plate, screw, or a combination thereof.

3. The improvement of claim 1, wherein the bio-resorbable components facilitate a limited degree of motion or mobility during or after healing.

4. The improvement of claim 3, wherein the limited degree of motion or mobility is controlled by the flexibility of the bioresorbable components.

5. The improvement of claim 4, wherein the flexibility of the components is due in part to the modulus of elasticity of the bioresorbable components.

6. The improvement of claim 4, wherein the flexibility of the components is due in part to the thickness or other physical attribute of the bioresorbable components.

7. The improvement of claim 3, wherein the limited degree of motion or mobility is controlled by the rate of resorbtion of the components.

8. The improvement of claim 1, wherein the arthroplasty device includes allograft.
